(19) United States
(12) Patent Application Publication
(10) Pub. No.: US 2020/0046703 A1
(43) Pub. Date: Feb. 13, 2020

(54) COMPOSITIONS AND METHODS FOR TREATMENT OF THE SIDE-EFFECTS ASSOCIATED WITH ADMINISTRATION OF THERAPEUTIC AGENTS

(71) Applicant: ASYMMETRIC THERAPEUTICS, LLC, Unadilla, NY (US)

(72) Inventor: John P. FORD, Unadilla, NY (US)

(21) Appl. No.: 16/379,057

(22) Filed: Apr. 9, 2019

Related U.S. Application Data
(60) Provisional application No. 62/716,544, filed on Aug. 9, 2018.

(51) Int. Cl. A61K 31/519; A61K 45/06; A61P 17/00; A61K 9/00

(52) U.S. Cl. CPC A61K 31/519; A61K 45/06; A61P 35/00; A61K 9/0014; A61P 17/00

(57) ABSTRACT

A protective formulation for treatment of at least one side-effect associated with administration of at least one therapeutic agent includes a therapeutic amount of oxypurinol. A method for treatment of at least one side-effect associated with administration of at least one therapeutic agent to a patient includes applying or locally delivering a protective formulation including a therapeutic amount of oxypurinol to a site of protection of the patient. In some embodiments, the side-effect is hand-foot syndrome, the therapeutic agent includes a fluoropyrimidine, and the protective formulation is a topical formulation.

### CROSS-REFERENCE TO RELATED APPLICATIONS

This application claims priority to and the benefit of U.S. Provisional Patent Application No. 62/716,544 filed Aug. 9, 2018, which is hereby incorporated by reference in its entirety.

### FIELD OF THE INVENTION

The present embodiments are directed to the field of ameliorating the effect of drugs on the human body. More particularly, the present embodiments pertain to compositions and methods for treatment of the cutaneous side-effects to the hands and feet (“hand-foot syndrome”) and oral mucosa associated with the administration of cancer chemotherapeutic agents and other therapeutic agents that cause hand-foot syndrome.

### BACKGROUND OF THE INVENTION

Cancer treatment drugs, while effective at destroying a cancerous tumor, may also cause damage to normal tissues of the body. The normal tissues of the body most often affected by the side-effects of a cancer chemotherapeutic drug include the lining of the mouth, the lining of the intestine, and the hair. Symptoms associated with the deleterious effects of chemotherapeutic cancer drugs include hair loss, nausea, and vomiting. In addition, certain cancer chemotherapeutic drugs affect the hands and the feet in a manner known as hand-foot syndrome (HFS). The side-effects associated with the administration of cancer chemotherapeutic drugs can be debilitating and result in interruptions of the cancer chemotherapeutic drug treatment regimen. Other systemically-administered therapeutic agents may also cause HFS or other side-effects in various organs and tissues that are not involved in the disease being treated. Many of these agents interact with, and are metabolized by, complex metabolic pathways.

One family of therapeutic drugs is the fluoropyrimidines, which include, but are not limited to, capecitabine, carmofur, doxifluridine, floxuridine, 5-fluorouracil (5-FU), and tegafur. These therapeutic drugs are often used in cancer chemotherapy. Among the poorly understood side-effects of fluoropyrimidine administration, the physiology of HFS is perhaps the most obscure. HFS is also a common side effect of certain non-pyrimidine agents, such as, for example, sunitinib, liposomal doxorubicin, and oxaliplatin.

HFS usually starts with numbness, tingling, redness, and painless swelling of the hands and/or feet. Grade 1 HFS is characterized by any of numbness, dysesthesia/paresthesia, tingling, and/or painless swelling or erythema of the distal extremities. Grade 2 is defined as a painful rash or erythema of the palms of the hands and/or the soles of the feet and/or discomfort affecting the patient's activities of daily living. Grade 3 HFS is defined as moist desquamation, ulceration, and blistering or severe pain of the hands and/or feet and/or severe discomfort that causes the patient to be unable to work or perform activities of daily living.

HFS is progressive with dose and duration of exposure to fluoropyrimidines. The pathophysiology of HFS is as yet unknown and variously ascribed to metabolites of 5-FU, local drug accumulation, increased levels of anabolic enzymes in the affected tissues, and various other factors [see, for example, Childress et al., “Cutaneous Hand and Foot Toxicity Associated With Cancer Chemotherapy”, Amer. J. Clinical Oncology, Vol. 26, pp. 435-436 (2003); Elasmar et al., “Case Report: Hand-Foot Syndrome Induced by the Oral Fluoropyrimidine 5-1”, Jpn. J. Clin. Oncol., Vol. 31, pp. 172-174 (2001); and Fischel et al., “Experimental arguments for a better understanding of hand-foot syndrome under capecitabine”, Proc. Amer. Ass'n Cancer Res., Vol. 45, p. 487 (abstract #2119) (March 2004)].

Capecitabine is currently in wide use in human cancer treatment. Capecitabine, however, has a major dose limiting toxicity with respect to HFS. A number of agents are somewhat effective in protecting patients from the toxicity of 5-FU, which is the active form of capecitabine. Among these is uracil. Uracil competes with 5-FU for activation by the salvage enzymes thymidine phosphorylase and uridine phosphorylase.

Various other attempts have been made to lessen or to eliminate the symptoms associated with the administration of 5-FU. One such approach to mitigate the toxicity of 5-FU is to combine a 5-FU precursor drug with other agents such as oral oxonic acid and 5-chloro-2,4-dihydroxypyridine in the case of the combination drug tegafur/gimeracil/oteracil (S-1). These agents have their own toxicities, including gastrointestinal (GI) toxicity [see, for example, Hoff, “The tegafur-based dihydropyrimidine dehydrogenase inhibitory fluoropyrimidines, UFT/leucovorin (ORZWL) and S-1: a review of their clinical development and therapeutic potential”, Investigational New Drugs, Vol. 18, pp. 331-342, (2000)]. Other agents, such as steroids, have been administered to patients to alleviate the suffering associated with the side-effects of cancer treatment using chemotherapy. The effect associated with the use of these other agents to alleviate suffering is often unsuccessful. As a consequence, the “treatment” is to lower the dose of 5-FU, a treatment intervention that decreases the anticancer activity of the drug being used to treat the cancer.

Another problem associated with these other agents is that agents such as steroids and others used to alleviate the side-effects of cancer drugs may be toxic to other tissues. Such tissue toxicity produces additional unwanted side-effects.

A third problem associated with drugs administered to alleviate the side-effects of cancer therapy is that the drug used to alleviate the side-effects caused by the cancer drug may interfere with the activity of the cancer drug, resulting in diminished effectiveness for destroying the targeted cancerous tumor.

The doubling time of normal skin is about 27 days [see, for example, Squier et al., ed., Human Oral Mucosa: Development, Structure, and Function, Wiley-Blackwell, p. 29 (2011)]. This fact has made it difficult in vitro to model agents to attenuate the HFS toxicity.

Allopurinol has been used in humans clinically in attempts to prevent 5-FU toxicity [see, for example, Howell et al., “Modulation of 5-Fluorouracil Toxicity by Allopurinol in Man”, Cancer, Vol. 48, pp. 1281-1289, (1981)]. Howell found that at the maximally-tolerated oral dose of 800 mg/day, allopurinol and thus also its major active metabolite oxypurinol does not prevent the mucositis toxicity of 5 μM of 5-FU. Oxypurinol, however, at about a 30-fold excess was found to be able to completely protect marrow cells from 5-FU toxicity in patients. This difference is likely due to differences in cell replication and metabolism between the two tissues.

Previous work also showed that oxypurinol inhibits the de novo synthesis of pyrimidines by inhibiting orotidine monophosphate decarboxylase [see, for example, Garewal et al., “Lack of Inhibition by Oxipurinol of 5-FU Toxicity Against Human Tumor Cell Lines”, Cancer Treatment Reports, Vol. 67, pp 495-498 (1983)]. Additionally, human gingival cells, in contrast to the columnar cells, metabolize pyrimidines differently [see, for example, Vanden Heuvel et al., “Differential nucleobase protection against 5-fluorouracil toxicity for squamous and columnar cells: implication for tissue function and oncogenesis”, Invest. New Drugs, Vol. 33, pp. 1003-1011 (2015)].

Allopurinol and oxypurinol have in the past been used in topical treatments intended to prevent skin cancer development in mice [see, for example, WO94/05291, entitled “Skin Cancer Treatment Compositions Containing Dimethyl Sulphone and Oxypurinol or Allopurinol”, published Mar. 17, 1994 to Salim]. Salim found allopurinol in combination with methylsulfonylmethane was a more potent protective than oxypurinol in combination with methylsulfonylmethane, although neither was very effective as a cancer preventative.

In U.S. Pat. No. 8,623,878, entitled “Use of allopurinol for the treatment of hand foot skin reaction” and issued on Jan. 7, 2014, Rodemer discloses the use of allopurinol to treat HFS. In fact, allopurinol has been used to prevent HFS from 5-FU, but at 3%, allopurinol did not achieve an adequate therapeutic benefit in clinical trials [see “Effectiveness Allopurinol Topical Agent Prevention Capecitabine-induced Hand-foot Syndrome”, Clinical Trial NCT01609166, completed December 2012], and a larger trial has not been started to date.

### BRIEF DESCRIPTION OF THE INVENTION

An assessment of agents to attenuate 5-FU-induced HFS attempted to control for inconsistent in vitro results from the altered cell metabolism of different cell growth conditions. Standard in vitro growth conditions with rapidly-proliferating cells, generally, do not replicate the growth patterns of skin cells/tissue clinically subject to HFS drug toxicity. To obtain an optimal model, human primary epithelial keratinocyte (HPEK) cells and immortalized normal epithelial keratinocyte (NHEK) cells, both of which are keratin-producing cell lines, were used. Both cell types were tested for protection form 5-FU toxicity, at confluence, a time during which the cells undergo minimal growth.

The results unexpectedly show that oxypurinol is a very effective preventive of 5-FU-induced toxicity to human keratinocytes in vitro, in contrast to allopurinol. The protective effect was most pronounced at an oxypurinol dose that caused a decrease in cell growth in the absence of 5-FU. The addition of 5-FU caused a negligible increase in cell death compared to culture in oxypurinol without the 5-FU. In fact, NHEK cells demonstrated an increase in cellular toxicity in the presence of increasing allopurinol concentrations.

These results are consistent with the known relatively more rapid replication of cells of the hands and feet [see, for example, Farr et al., “Palmar-Plantar Erythrodysesthesia Associated with Chemotherapy and Its Treatment”, Case Reports in Oncology, Vol. 4, pp. 229-235 (2011)] in comparison to the rest of the skin. Without wishing to be bound by theory, it is believed that by slowing cell replication, oxypurinol is creating a growth pattern more like the slower cell replication of the cells of the non-palmar and non-plantar skin that are not subject to 5-FU toxicity.

On this basis, it is expected that oxypurinol is of benefit in a protective treatment against all agents that cause a toxicity to the hands and feet but not to the relatively still slower-growing non-palmar and non-plantar human skin.

In an embodiment, a protective formulation for treatment of at least one side-effect associated with administration of at least one therapeutic agent includes a therapeutic amount of oxypurinol.

In another embodiment, a method for treatment of at least one side-effect associated with administration of at least one therapeutic agent to a patient includes applying or locally delivering a protective formulation including a therapeutic amount of oxypurinol to a site of protection of the patient.

Other features and advantages of the present invention will be apparent from the following more detailed description, taken in conjunction with the accompanying drawings, which illustrate, by way of example, the principles of the invention.

### BRIEF DESCRIPTION OF THE DRAWINGS

FIG. 1 shows the relative growth of primary human epidermal keratinocyte (HPEK) cells in the presence of certain concentrations of oxypurinol or allopurinol in the presence of 5-FU on a semilogarithmic scale.

FIG. 2 shows the relative growth of normal human epidermal keratinocyte (NHEK) cells in the presence of certain concentrations of oxypurinol or allopurinol in the presence of 5-FU on a semilogarithmic scale.

Wherever possible, the same reference numbers will be used throughout the drawings to represent the same parts.

### DETAILED DESCRIPTION OF EXEMPLARY EMBODIMENTS

Provided are compositions and methods for the treatment of the side-effects associated with the administration of therapeutic agents.

Embodiments of the present disclosure, for example, in comparison to concepts failing to include one or more of the features disclosed herein, reduce or prevent a side-effect associated with the administration of one or more therapeutic agents to a patient, reduce or prevent a side-effect associated with the administration of one or more cancer chemotherapeutic agents to a patient, reduce the likelihood or severity of occurrence of hand-foot syndrome (HFS) associated with a therapeutic treatment, reduce the likelihood or severity of occurrence of HFS associated with chemotherapy treatment, reduce the likelihood or severity of occurrence of oral stomatitis associated with a therapeutic or chemotherapy treatment in a patient, reduce the likelihood or severity of occurrence of gastrointestinal toxicity associated with a therapeutic or chemotherapy treatment in a patient, or combinations thereof.

In exemplary embodiments, a protective formulation includes a therapeutic amount of oxypurinol. The protective formulation treats at least one side-effect associated with administration of at least one therapeutic agent. In some embodiments, the therapeutic agent is a cancer chemotherapeutic agent. In some embodiments, the side-effect is HFS. In some embodiments, the therapeutic agent includes a fluoropyrimidine. In some embodiments, the fluoropyrimidine includes capecitabine, carmofur, doxifluridine, floxuridine, 5-fluorouracil (5-FU), and/or tegafur. In some embodiments, the cancer chemotherapeutic agent is a fluoropyrimidine. In some embodiments, the therapeutic agent includes a non-pyrimidine agent. In some embodiments, the non-pyrimidine agent includes sunitinib, liposomal doxorubicin, and/or oxaliplatin. In some embodiments, the therapeutic agent is a cancer chemotherapeutic agent. In some embodiments, the protective formulation is a topical formulation.

In view of the unmet clinical need, oxypurinol (the active form of allopurinol, an agent in long use in the treatment of gout) was tested in tissue culture for two different human keratinocyte cell strains, primary human epidermal keratinocyte (HPEK) cells and normal human epidermal keratinocyte (NHEK) cells, as well as primary human gingival epithelial (HGEP) cells. NHEK cells are SV40 immortalized normal keratinocytes.

The present results with NHEK and HPEK show that there is a progressive decrease in the toxicity of 10 μM of 5-FU with increasing doses of oxypurinol up to the maximum in vitro solubility of about 3 mM of oxypurinol. There is no evidence of oxypurinol toxicity.

As a topical agent, oxypurinol may be formulated in a hydrophobic ointment at about 1%, by weight, (about 60 mM) that is well-tolerated. This constitutes about a 10,000-fold local excess of oxypurinol over 5-FU. In the extremely unlikely event that the oxypurinol is quantitatively absorbed, the daily body oxypurinol burden would be about 10 mg or about 1/30 of the standard dosing of allopurinol for gout. Also, the systemic level of oxypurinol would still be 10/800 or 1/80 of a systemic oxypurinol dose that was not effective in protecting tumor cells for 5-FU toxicity.

Oxypurinol has about a 2% to 3% incidence of rash. It is nearly unheard of in the literature for any agent applied to intact non-mucosal skin to have a serious skin reaction [see, for example, Sachs et al., “Anaphylaxis and toxic epidermal necrolysis or Stevens-Johnson syndrome after nonmucosal topical drug application: fact or fiction?”, Allergy, Vol. 62, pp. 877-883 (2007)], indicating that a topical composition should be given prophylactically.

In some embodiments, compositions and methods include oxypurinol as an active ingredient in a protective formulation.

In some embodiments, compositions and methods include oxypurinol as an active ingredient in a protective formulation that is free of adenine and free of uracil.

In some embodiments, the protective formulation is formulated for topical application to skin, the protective formulation can usefully be formulated as a topical formulation. Appropriate topical formulations may include, but are not limited to, an ointment, a cream, a lotion, a paste, an aerosol spray, a roll-on liquid, stick, or pad, or an aerosol foam (mousse) composition. In some embodiments, compositions and methods include topical delivery of a protective formulation including oxypurinol as an active ingredient as described in U.S. Pat. No. 9,084,788, entitled “Compositions and methods for treating and preventing dermatoses” and issued to Ford on Jul. 21, 2015, which discloses compositions and methods for topical administration.

The exact formulation of the protective formulation will depend upon the identity of the tissue desired to be protected. Pharmaceutical formulation is a well-established art [see, for example, Allen ed., Remington: The Science and Practice of Pharmacy, 22nd ed., Pharmaceutical Press (2012); Allen, Ansel's Pharmaceutical Dosage Forms and Drug Delivery Systems, 11th ed., Wolters Kluwer (2017); and Rowe et al., ed., Handbook of Pharmaceutical Excipients, 6th ed., Pharmaceutical Press (2009)].

Appropriate topical formulations may, for example, be anhydrous, aqueous, or water-in-oil or oil-in-water emulsions. Appropriate topical formulations may further include one or more pharmaceutically acceptable carriers or excipients and various skin actives. Amounts of the carrier may range from about 1 to about 99%, preferably from about 5 to about 70%, optimally from about 10 to about 40% by weight. Among useful carriers are emollients, water, inorganic powders, foaming agents, emulsifiers, fatty alcohols, fatty acids, and combinations thereof.

Emollients may be selected from polyols, esters and hydrocarbons. Polyols suitable for the invention may include propylene glycol, dipropylene glycol, polypropylene glycol, polyethylene glycol, sorbitol, hydroxypropyl sorbitol, hexylene glycol, 1,3-butylene glycol, 1,2,6-hexanetriol, glycerin, ethoxylated glycerin, propoxylated glycerin, xylitol and mixtures thereof.

Esters useful as emollients include alkyl esters of fatty acids having 10 to 20 carbon atoms. Methyl, isopropyl, and butyl esters of fatty acids may be useful. Examples include hexyl laurate, isohexyl laurate, isohexyl palmitate, isopropyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, lauryl lactate, myristyl lactate, and cetyl lactate. Particularly preferred are C12-C15 alcohol benzoate esters.

Esters useful as emollients may also include alkenyl esters of fatty acids having 10 to 20 carbon atoms, such as, for example, oleyl myristate, oleyl stearate and oleyl oleate. Esters useful as emollients may also include ether-esters such as fatty acids esters of ethoxylated fatty alcohols.

Esters useful as emollients may also include polyhydric alcohol esters, such as, for example, ethylene glycol mono- and di-fatty acid esters, diethylene glycol mono- and di-fatty acid esters, polyethylene glycol (200 6000) mono- and di-fatty acid esters, polyglycerol poly-fatty esters, ethoxylated glyceryl monostearate, 1,3-butylene glycol monostearate, 1,3-butylene glycol distearate, polyoxyethylene polyol fatty acid ester, sorbitan fatty acid esters, and/or polyoxyethylene sorbitan fatty acid esters.

Esters useful as emollients may additionally include wax esters, such as, for example, beeswax, spermaceti, myristyl myristate, and/or stearyl stearate.

Esters useful as emollients still further may include sterol esters, such as, for example, cholesterol fatty acid esters.

Appropriate hydrocarbon carriers may include mineral oil, polyalphaolefins, petrolatum, isoparaffin, polybutenes, and/or mixtures thereof.

Inorganic powders may also useful as carriers in topical formulations. Examples may include clays (such as, for example, Montmorillonite, Hectorite, Laponite and Bentonite), talc, mica, silica, alumina, zeolites, sodium sulfate, sodium bicarbonate, sodium carbonate, calcium sulfate, and/or mixtures thereof.

Appropriate topical formulations may also include aerosol propellants, serving as, or in addition to, carriers or excipients. Propellants may be based on volatile hydrocarbons such as propane, butane, isobutene, pentane, isopropane and mixtures thereof. Phillips Petroleum Company (Bartlesville, Okla.) may be a source of such propellants under trademarks including A3, A32, A51, and/or A70. Halocarbons including fluorocarbons may further widely be employed propellants.

Appropriate topical formulations for administration to the skin may include emulsifiers, either serving as, or in addition to, carriers and/or excipients.

Appropriate emulsifiers may be selected from nonionic, anionic, cationic, and/or amphoteric emulsifying agents. Appropriate emulsifiers may range in amount anywhere from about 0.1 to about 20% by weight.

Appropriate nonionic emulsifiers may include alkoxylated compounds based on C10-C22 fatty alcohols and acids and sorbitan. Appropriate materials may be available, for instance, under the Neodol trademark (Shell Oil Company, Houston, Tex.), as copolymers of polyoxypropylenepolyoxyethylene sold under the Pluronic trademark (BASF Corporation, Ludwigshafen, Germany), and/or as alkyl polyglycosides available from the Henkel Corporation (Dusseldorf, Germany).

Appropriate anionic type emulsifiers may include fatty acid soaps, sodium lauryl sulfate, sodium lauryl ether sulfate, alkyl benzene sulfonate, mono- and di-alkyl acid phosphates, sarcosinates, taurates, and/or sodium fatty acyl isethionate.

Appropriate amphoteric emulsifiers may include dialkylamine oxide and various types of betaines, such as, for example, cocamidopropyl betaine.

Appropriate topical formulations may also include preservatives, such as, for example, methyl paraben and propyl paraben, which are useful to prevent microbial contamination.

In some embodiments, compositions and methods include oral delivery of a protective formulation including oxypurinol as an active ingredient as described in U.S. Pat. No. 9,119,855, entitled “Compositions and methods for treatment of the side-effects associated with administration of cancer chemotherapeutic agents” and issued to Ford on Sep. 1, 2015, which discloses compositions and methods for oral administration.

In some embodiments, the protective formulation is applied or delivered locally to the site of protection at a concentration that would salvage the tumor cells from the toxicity of the cancer chemotherapeutic agent if supplied to the tumor cells at that concentration.

In some embodiments, each active ingredient may be present in the protective formulation in a weight percentage of at least 0.01%, 0.05%, 1.0%, 1.5%, 2.0%, 2.5%, 3.5%, 4.0%, 4.5%, 5.0%, 10%, 15%, 20%, 25%, 30%, 35%, 40%, 45%, even 50% or more, with intermediate values permissible, and is typically present to a weight/weight percentage of no more than about 50%, 45%, 40% 30%, 35%, 30%, 25%, 20%, 15%, 10%, 5%, 45%, 4.0%, 3.5%, 3.0%, 2.5%, 2.0%, 1.5%, 1.0%, and even, at times, to a weight/weight percentage of no more than about 0.05%, even as little as 0.01%, with intermediate values permissible.

In some embodiments, the protective formulation is applied or delivered locally to the site of protection to provide a sustained concentration of the active ingredient of 0.03 mM to 3 mM, alternatively at least 0.03 mM, alternatively 0.03 mM to 0.1 mM, alternatively at least 0.1 mM, alternatively 0.1 mM to 0.3 mM, alternatively at least 0.3 mM, alternatively 0.3 mM to 1 mM, alternatively at least 1 mM, alternatively 1 mM to 3 mM, alternatively at least 3 mM, alternatively 3 mM or less, alternatively 1 mM or less, alternatively 0.3 mM or less, alternatively 0.1 mM or less, or any value, range, or sub-range therebetween.

In some embodiments, the active ingredient includes oxypurinol. In some embodiments, oxypurinol is the primary active ingredient in the protective formulation. In some embodiments, oxypurinol is the only active ingredient in the protective formulation. In some embodiments, the protective formulation is free or substantially free of allopurinol, adenine, and/or uracil. Substantially free, as used herein, refers to an amount of less than 0.001 mM of a compound in a composition.

Oxypurinol is the major active metabolite of allopurinol and is an inhibitor of xanthine oxidase that is cleared by the kidneys. Allopurinol and oxypurinol have the following structures:

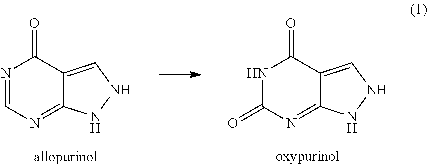 [SMILES: O=c1ncnc2[nH][nH]cc1-2 ; O=c1nc2[nH][nH]cc-2c(=O)[nH]1]

HGEP cells provide a model in vitro gingival system for studying toxicity, whereas HPEK cells and NHEK cells provide model in vitro skin systems for studying toxicity. For evaluation of HFS protection, HPEK cells and NHEK cells are a better in vitro model than HGEP cells, because they produce keratin, like the cells of the hands and feet. HGEP cells are not keratin-producing cells.

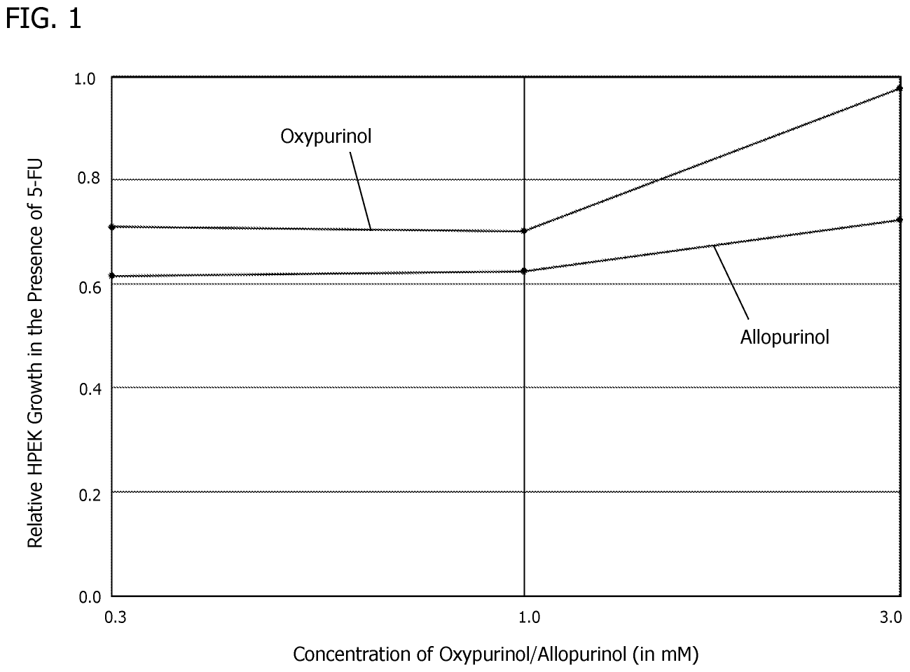

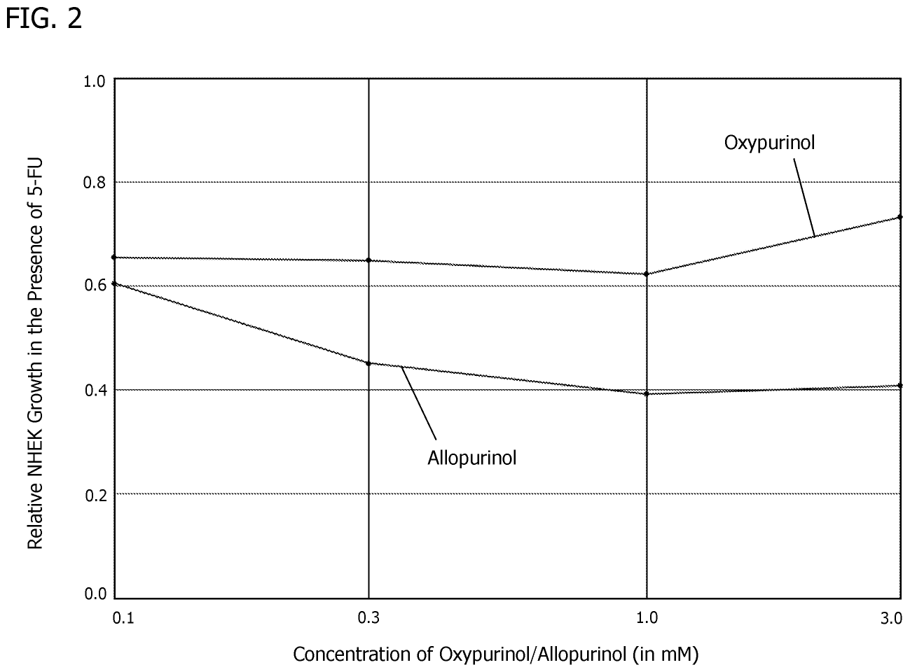

Since oxypurinol is a metabolite of allopurinol, both would be expected to have a similar effect on cells. FIG. 1 and FIG. 2, however, show the unpredictable, surprising, and unexpected result that oxypurinol is superior to allopurinol in protecting keratin-producing cells from 5-FU toxicity.

The presence of the oxypurinol, in the absence of adenine and uracil, has a positive effect on the viability of HGEP cells, HPEK cells, and NHEK cells in the presence of 5-FU, as shown in Table 1 through Table 6.

For proliferating HGEP cells, the presence of oxypurinol has a negligible effect when adenine or uracil is present at 0.1 mM in the presence of 5-FU, as shown in Table 1. Oxypurinol has a positive effect on HGEP cells when the adenine or uracil is present at 0.3 mM in the presence of 5-FU, but the co-presence of the uracil still negatively affects the viability of the HGEP cells. The co-presence of adenine at 0.3 mM for HGEP cells in the presence of 5-FU and oxypurinol was the only instance of the co-presence of adenine or uracil showing a positive effect. For HPEK cells, the co-presence of adenine or uracil hinders the protective effect of oxypurinol up to oxypurinol concentrations of 0.3 mM, as shown in Table 2, although adenine hinders more than uracil.

It is particularly noteworthy that the survival of confluent HPEK cells increased with increasing concentration of oxypurinol together with 10 μM of 5-FU from 0.64 to 0.72 to 0.75 to 0.81 as the concentration of oxypurinol increased from 0 mM to 0.3 mM to 1 mM to 3 mM, respectively, as shown in Table 3. At least as interesting, the relative viability of the same cells in the presence of oxypurinol alone decreased between 1 mM and 3 mM from 1.07 to 0.835. Allopurinol also decreased the relative viability of the same cells by a similar amount with increase from 1 mM to 3 mM. Computing the growth of the cells in the presence of oxypurinol at 3 mM with and without 5-FU, one detects a 97.5% preservation of growth. The addition of the 5-FU with oxypurinol at 3 mM confers virtually no additional toxicity, in contrast to the 5-FU growth effect with allopurinol at 3 mM.

Similarly, the survival of the confluent NHEK cells increased with increasing concentration of oxypurinol together with 10 μM of 5-FU from 0.42 to 0.58 to 0.59 to 0.60 to 0.72 as the concentration of oxypurinol increased from 0 mM to 0.1 mM to 0.3 mM to 1 mM to 3 mM, respectively, as shown in Table 4. In contrast, increasing the allopurinol concentration in the presence of 5-FU from 0.1 mM to 0.3 mM to 1 mM to 3 mM decreased the survival of NHEK cells from 0.63 to 0.51 to 0.44 and 0.44, respectively, as shown in Table 4.

Without wishing to be bound by theory, the oxypurinol may be limiting uptake of nucleobase (including 5-FU), as the application of either adenine or uracil alone exerts marked growth-enhancing effects and suggests that nucleobase uptake may be growth-limiting. The sensitivity of tissues to 5-FU exposure is related to their growth fraction. Thus, GI cells and marrow cells are very sensitive, but lung cells are much less so. If oxypurinol is decreasing the growth fraction of cells, it perhaps makes the cells more like those of a resistant tissue.

As noted previously, human epidermal keratinocytes have a slow growth cycle in vivo of about 27 days, which makes their study in vitro difficult. In contrast, HPEK and NHEK cells have a much faster growth cycle in vitro of about 1 day. The beneficial protection afforded by oxypurinol is greater at slower growth rates and therefore is expected to be greater in vivo on the palms of the hands and the soles of the feet than what was observed in vitro with HPEK and NHEK.

Although not everyone under a fluoropyrimidine treatment develops HFS, the negligible toxicity of locally therapeutic doses of oxypurinol indicates a significant benefit to proactive administration topically and locally of a protective formulation including a therapeutic amount of oxypurinol to the palms of the hands and the soles of the feet prior to and during administration of a fluoropyrimidine to prevent or lessen the severity of HFS caused by fluoropyrimidine toxicity. The therapeutic dose includes a therapeutic amount of the oxypurinol sufficient to protect cells of the palms of the hands and the soles of the feet from fluoropyrimidine toxicity.

In some embodiments, the protective formulation including a therapeutic amount of oxypurinol is administered topically and locally to the palms of the hands and the soles of the feet prior to and during administration of a therapeutic agent to prevent or lessen the severity of HFS caused by toxicity of the therapeutic agent. The therapeutic dose includes a therapeutic amount of the oxypurinol sufficient to protect cells of the palms of the hands and the soles of the feet from toxicity of the therapeutic agent.

### EXAMPLES

The invention is further described in the context of the following examples which are presented by way of illustration, not of limitation.

The efficacy of certain compounds and combinations of compounds as protective agents when co-administered with 5-FU was tested in cell culture.

### Example 1

In one set of experiments, the results of which are shown in Table 1, proliferating HGEP cells, commonly used to assess oral mucosal toxicity, were exposed to the clinically-relevant dose of 10 μM of 5-FU and different concentrations of oxypurinol, either alone or with different concentrations of adenine or uracil, using standard tissue culture methods and incubated at 37° C. Oxypurinol concentrations of 0.03 mM, 0.1 mM, and 0.3 mM were tested. Adenine and uracil concentrations of 0.1 mM and 0.3 mM were tested. The relative viability of the cells was determined after 120 hours.

TABLE 1
Proliferating HGEP cell viability

| Treatment | AVG | SEM | p-value¹ | Stats² |
|---|---|---|---|---|
| Control (0.2% DMSO) [no treatment or 5-FU] | 1.0000 | 0.0099 | <.0001 | A |
| No treatment [5-FU (10 μM)] | 0.6217 | 0.0144 | 1 | DE |
| Oxypurinol (0.03 mM) [no 5-FU] | 0.8792 | 0.0170 | <.0001 | ABC |
| Oxypurinol (0.1 mM) [no 5-FU] | 0.7134 | 0.0471 | 0.5772 | BCD |
| Oxypurinol (0.3 mM) [no 5-FU] | 0.6367 | 0.0364 | 1 | DE |
| Oxypurinol (0.03 mM) [5-FU (10 μM)] | 0.6441 | 0.0238 | 1 | DE |
| Oxypurinol (0.1 mM) [5-FU (10 μM)] | 0.7540 | 0.0136 | 0.1375 | BCD |
| Oxypurinol (0.3 mM) [5-FU (10 μM)] | 0.8925 | 0.0081 | <.0001 | AB |
| Adenine (0.1 mM) [5-FU (10 μM)] | 0.7549 | 0.0142 | 0.1323 | BCD |
| Oxypurinol (0.03 mM) + Adenine (0.1 mM) [5-FU (10 μM)] | 0.7798 | 0.0059 | 0.0401 | BCD |
| Oxypurinol (0.1 mM) + Adenine (0.1 mM) [5-FU (10 μM)] | 0.7699 | 0.0243 | 0.0662 | BCD |
| Oxypurinol (0.3 mM) + Adenine (0.1 mM) [5-FU (10 μM)] | 0.7752 | 0.0191 | 0.0507 | BCD |
| Adenine (0.3 mM) [5-FU (10 μM)] | 0.6389 | 0.0097 | 1 | DE |
| Oxypurinol (0.03 mM) + Adenine (0.3 mM) [5-FU (10 μM)] | 0.7353 | 0.0140 | 0.2893 | BCD |
| Oxypurinol (0.1 mM) + Adenine (0.3 mM) [5-FU (10 μM)] | 0.8963 | 0.0165 | <.0001 | AB |
| Oxypurinol (0.3 mM) + Adenine (0.3 mM) [5-FU (10 μM)] | 0.9853 | 0.0186 | <.0001 | A |
| Uracil (0.1 mM) [5-FU (10 μM)] | 0.6060 | 0.0243 | 1 | DE |
| Oxypurinol (0.03 mM) + Uracil (0.1 mM) [5-FU (10 μM)] | 0.6300 | 0.0520 | 1 | DE |
| Oxypurinol (0.1 mM) + Uracil (0.1 mM) [5-FU (10 μM)] | 0.7201 | 0.0657 | 0.4779 | BCD |
| Oxypurinol (0.3 mM) + Uracil (0.1 mM) [5-FU (10 μM)] | 0.6420 | 0.0713 | 1 | DE |
| Uracil (0.3 mM) [5-FU (10 μM)] | 0.5182 | 0.0502 | 0.4081 | E |
| Oxypurinol (0.03 mM) + Uracil (0.3 mM) [5-FU (10 μM)] | 0.6075 | 0.0606 | 1 | DE |
| Oxypurinol (0.1 mM) + Uracil (0.3 mM) [5-FU (10 μM)] | 0.6998 | 0.0576 | 0.7795 | CDE |
| Oxypurinol (0.3 mM) + Uracil (0.3 mM) [5-FU (10 μM)] | 0.7592 | 0.0415 | 0.1095 | BCD |

¹Dunnett's multi-comparison to 5-FU only (p < 0.05 considered significant)
²Tukey's multi-comparison (levels not connected by same letter significantly different (p < 0.05))

Still referring to Table 1, the viability of proliferating HGEP cells in the presence of 10 μM of 5-FU was only 62.2% compared to a control system without 5-FU. The presence of oxypurinol in the absence of 5-FU also had a negative effect on proliferating HGEP cell viability, although a much weaker one than 5-FU in the absence of oxypurinol. When applied in the presence of 5-FU, however, oxypurinol showed a protective effect for HGEP cells at concentrations of 0.1 mM and 0.3 mM.

In the presence of 0.1 mM of adenine or uracil, oxypurinol showed a negligible effect on the viability of proliferating HGEP cells in the presence of 5-FU. At the higher adenine or uracil concentration of 0.3 mM, oxypurinol at concentrations of 0.1 mM and 0.3 mM again showed a protective effect in the presence of 5-FU.

### Example 2

In another set of experiments, the results of which are shown in Table 2, proliferating HPEK cells, commonly used to assess skin tissue toxicity, were exposed to the clinically-relevant dose of 10 μM of 5-FU and different concentrations of oxypurinol, either alone or with different concentrations of adenine or uracil, using standard tissue culture methods and incubated at 37° C. Oxypurinol concentrations of 0.03 mM, 0.1 mM, and 0.3 mM were tested. Adenine and uracil concentrations of 0.1 mM and 0.3 mM were tested. The relative viability of the cells was determined after 120 hours.

TABLE 2
Proliferating HPEK cell viability

| Treatment | AVG | SEM | p-value¹ | Stats² |
|---|---|---|---|---|
| Control (0.2% DMSO) [no treatment or 5-FU] | 1.0000 | 0.0085 | <.0001 | AB |
| No treatment [5-FU (10 μM)] | 0.6507 | 0.0150 | 1 | DE |
| Oxypurinol (0.03 mM) [no 5-FU] | 1.0763 | 0.0054 | <.0001 | A |
| Oxypurinol (0.1 mM) [no 5-FU] | 0.7961 | 0.1060 | 0.0447 | CD |
| Oxypurinol (0.3 mM) [no 5-FU] | 1.0201 | 0.0100 | <.0001 | AB |
| Oxypurinol (0.03 mM) [5-FU (10 μM)] | 0.6903 | 0.0150 | 0.9992 | CDE |
| Oxypurinol (0.1 mM) [5-FU (10 μM)] | 0.7961 | 0.0109 | 0.0448 | CD |
| Oxypurinol (0.3 mM) [5-FU (10 μM)] | 0.8635 | 0.0139 | 0.0005 | BC |
| Adenine (0.1 mM) [5-FU (10 μM)] | 0.6214 | 0.0107 | 1 | DE |
| Oxypurinol (0.03 mM) + Adenine (0.1 mM) [5-FU (10 μM)] | 0.6764 | 0.0075 | 1 | DE |
| Oxypurinol (0.1 mM) + Adenine (0.1 mM) [5-FU (10 μM)] | 0.6325 | 0.0144 | 1 | DE |
| Oxypurinol (0.3 mM) + Adenine (0.1 mM) [5-FU (10 μM)] | 0.6982 | 0.0032 | 0.9923 | CDE |
| Adenine (0.3 mM) [5-FU (10 μM)] | 0.5985 | 0.0053 | 0.9795 | E |
| Oxypurinol (0.03 mM) + Adenine (0.3 mM) [5-FU (10 μM)] | 0.6306 | 0.0078 | 1 | DE |
| Oxypurinol (0.1 mM) + Adenine (0.3 mM) [5-FU (10 μM)] | 0.7002 | 0.0236 | 0.988 | CDE |
| Oxypurinol (0.3 mM) + Adenine (0.3 mM) [5-FU (10 μM)] | 0.7341 | 0.0123 | 0.6106 | CDE |
| Uracil (0.1 mM) [5-FU (10 μM)] | 0.6479 | 0.0124 | 1 | DE |
| Oxypurinol (0.03 mM) + Uracil (0.1 mM) [5-FU (10 μM)] | 0.6914 | 0.0428 | 0.9988 | CDE |
| Oxypurinol (0.1 mM) + Uracil (0.1 mM) [5-FU (10 μM)] | 0.7326 | 0.0522 | 0.6356 | CDE |
| Oxypurinol (0.3 mM) + Uracil (0.1 mM) + 5-FU (10 μM) | 0.7177 | 0.0492 | 0.8602 | CDE |
| Uracil (0.3 mM) [5-FU (10 μM)] | 0.6350 | 0.0444 | 1 | DE |
| Oxypurinol (0.03 mM) + Uracil (0.3 mM) [5-FU (10 μM)] | 0.6765 | 0.0363 | 1 | DE |
| Oxypurinol (0.1 mM) + Uracil (0.3 mM) [5-FU (10 μM)] | 0.7685 | 0.0461 | 0.1772 | CDE |
| Oxypurinol (0.3 mM) + Uracil (0.3 mM) [5-FU (10 μM)] | 0.7759 | 0.0320 | 0.1261 | CDE |

¹Dunnett's multi-comparison to 5-FU only (p < 0.05 considered significant)
²Tukey's multi-comparison (levels not connected by same letter significantly different (p < 0.05))

Still referring to Table 2, the viability of proliferating HPEK cells in the presence of 10 μM of 5-FU was only 65.1% compared to a control system without 5-FU. The presence of oxypurinol over the range of 0.03 mM to 0.3 mM in the absence of 5-FU had a minimal effect on proliferating HPEK cell viability. When applied in the presence of 5-FU, oxypurinol showed a protective effect for proliferating HPEK cells at concentrations of 0.1 mM and 0.3 mM. In the presence of 0.1 mM of adenine or uracil, oxypurinol showed a negligible effect on the viability of proliferating HPEK cells in the presence of 5-FU. At a higher adenine or uracil concentration of 0.3 mM, oxypurinol at concentrations of 0.1 mM and 0.3 mM showed a slight protective effect in the presence of 5-FU.

### Example 3

In yet another set of experiments, the results of which are shown in Table 3, confluent HPEK cells were exposed to the clinically-relevant dose of 10 μM of 5-FU and different concentrations of oxypurinol or allopurinol, either alone or with different concentrations of adenine, using standard tissue culture methods and incubated at 37° C. Oxypurinol and allopurinol concentrations of 0.3 mM, 1.0 mM, and 3.0 mM, which are ten times higher than those used in the first set of experiments, were tested. An adenine concentration of 0.3 mM, which was the higher concentration used in the first set of experiments, was tested. The relative viability of the cells was determined after 120 hours.

TABLE 3
Confluent HPEK cell viability

| Treatment | AVG | SEM | Stats¹ | Stats² |
|---|---|---|---|---|
| Control [no treatment or 5-FU] | 1.0000 | 0.0240 | D | A |
| No treatment [5-FU (10 μM)] | 0.6421 | 0.0291 | G | C |
| Oxypurinol (0.3 mM) [no 5-FU] | 1.0183 | 0.0406 | D | |
| Oxypurinol (1.0 mM) [no 5-FU] | 1.0706 | 0.0236 | CD | |
| Oxypurinol (3.0 mM) [no 5-FU] | 0.8351 | 0.0154 | E | |
| Allopurinol (0.3 mM) [no 5-FU] | 1.1870 | 0.0299 | BC | |
| Allopurinol (1.0 mM) [no 5-FU] | 1.2411 | 0.0498 | AB | |
| Allopurinol (3.0 mM) [no 5-FU] | 1.0410 | 0.0196 | CD | |
| Adenine (0.3 mM) [no 5-FU] | 1.3784 | 0.0193 | A | |
| Uracil (0.3 mM) [no 5-FU] | 1.3781 | 0.0216 | A | |
| Oxypurinol (0.3 mM) [5-FU (10 μM)] | 0.7226 | 0.0060 | EFG | C |
| Oxypurinol (1.0 mM) [5-FU (10 μM)] | 0.7509 | 0.0180 | EFG | BC |
| Oxypurinol (3.0 mM) [5-FU (10 μM)] | 0.8147 | 0.0381 | E | BC |
| Allopurinol (0.3 mM) [5-FU (10 μM)] | 0.7303 | 0.0060 | EFG | B |
| Allopurinol (1.0 mM) [5-FU (10 μM)] | 0.7748 | 0.0053 | EFG | BC |
| Allopurinol (3.0 mM) [5-FU (10 μM)] | 0.7528 | 0.0203 | EFG | BC |
| Adenine (0.3 mM) [5-FU (10 μM)] | 0.7456 | 0.0196 | EFG | BC |
| Uracil (0.3 mM) [5-FU (10 μM)] | 0.6482 | 0.0336 | FG | C |
| Oxypurinol (0.3 mM) + Adenine (0.3 mM) [5-FU (10 μM)] | 0.7821 | 0.0281 | EFG | BC |
| Oxypurinol (1.0 mM) + Adenine (0.3 mM) [5-FU (10 μM)] | 0.7986 | 0.0400 | EF | B |
| Oxypurinol (3.0 mM) + Adenine (0.3 mM) [5-FU (10 μM)] | 0.7998 | 0.0412 | E | B |
| Allopurinol (0.3 mM) + Adenine (0.3 mM) [5-FU (10 μM)] | 0.7928 | 0.0366 | EFG | B |
| Allopurinol (1.0 mM) + Adenine (0.3 mM) [5-FU (10 μM)] | 0.8178 | 0.0340 | E | B |
| Allopurinol (3.0 mM) + Adenine (0.3 mM) [5-FU (10 μM)] | 0.7726 | 0.0153 | EFG | BC |

¹Tukey's multi-comparison (levels not connected by same letter significantly different (p < 0.05))

Still referring to Table 3, the viability of confluent HPEK cells in the presence of 10 μM of 5-FU was only 65.1% compared to a control system without 5-FU. The presence of oxypurinol in the absence of 5-FU also had a neutral effect on confluent HPEK cell viability at 0.3 mM, a slightly positive effect at 1.0 mM, and a slightly negative effect at 3.0 mM. Similarly, the presence of allopurinol in the absence of 5-FU also had the strongest positive effect on confluent HPEK cell viability at 1.0 mM but showed positive effects also at 0.3 mM and 3.0 mM. The presence of adenine and uracil at 0.3 mM separately in the absence of 5-FU had a strong positive effect on confluent HPEK cell viability, stronger than the strongest positive effects for oxypurinol and allopurinol alone.

Still referring to Table 3, when applied in the presence of 5-FU, both oxypurinol and allopurinol showed a protective effect for confluent HPEK cells over the tested concentration range of 0.3 mM to 3.0 mM. The protective effect increased with increasing oxypurinol concentration. In contrast, allopurinol had a maximum protective effect at 1.0 mM. The additional presence of 0.3 mM of adenine slightly increased the protective effect of oxypurinol at 0.3 mM and 1.0 mM, but had a negligible effect on the viability of confluent HPEK cells at 3.0 mM of oxypurinol. The additional presence of 0.3 mM of adenine slightly increased the protective effect of allopurinol at 0.3 mM, 1.0 mM, and 3.0 mM.

### Example 4

In yet another set of experiments, the results of which are shown in Table 4, confluent NHEK cells were exposed to the clinically-relevant dose of 10 μM of 5-FU and different concentrations of sodium hydroxide, oxypurinol, or allopurinol using standard tissue culture methods and incubated at 37° C. Sodium hydroxide concentrations of 1 mM, 2 mM, 3 mM, and 4 mM were tested. Oxypurinol and allopurinol concentrations of 0.1 mM, 0.3 mM, 1.0 mM, and 3.0 mM were tested, both in the presence and in the absence of 10 μM of 5-FU. The relative viability of the cells was determined after 120 hours. The sodium hydroxide trials were only performed to serve as a control.

Still referring to Table 4, the viability of confluent NHEK cells in the presence of 10 μM of 5-FU was only 41.6% compared to a control system without 5-FU. The presence of oxypurinol over the range of 0.1 mM to 3.0 mM in the absence of 5-FU had a small negative effect on confluent NHEK cell viability that decreased with increasing oxypurinol concentration. When applied in the presence of 5-FU, oxypurinol showed a protective effect for confluent NHEK cells over the tested concentration range, with the greatest protection being at the highest tested concentration of 3.0 mM.

TABLE 4
Confluent NHEK cell viability

| Treatment | AVG | SEM | Stats¹ |
|---|---|---|---|
| Control [no treatment or 5-FU] | 1.0000 | 0.0139 | CDE |
| No treatment [5-FU (10 μM)] | 0.4165 | 0.0059 | N |
| NaOH (4 mM) [no 5-FU] | 0.9286 | 0.0245 | EFG |
| Oxypurinol (0.1 mM) [no 5-FU] | 0.8884 | 0.0255 | G |
| Oxypurinol (0.3 mM) [no 5-FU] | 0.9059 | 0.0152 | FG |
| Oxypurinol (1.0 mM) [no 5-FU] | 0.9580 | 0.0298 | EFG |
| Oxypurinol (3.0 mM) [no 5-FU] | 0.9784 | 0.0085 | DEF |
| Allopurinol (0.1 mM) [no 5-FU] | 1.0426 | 0.0227 | BCD |
| Allopurinol (0.3 mM) [no 5-FU] | 1.1285 | 0.0051 | A |
| Allopurinol (1.0 mM) [no 5-FU] | 1.1137 | 0.0034 | AB |
| Allopurinol (3.0 mM) [no 5-FU] | 1.0640 | 0.0025 | ABC |
| Oxypurinol (0.1 mM) [5-FU (10 μM)] | 0.5818 | 0.0087 | IJK |
| Oxypurinol (0.3 mM) [5-FU (10 μM)] | 0.5881 | 0.0142 | IJK |
| Oxypurinol (1.0 mM) [5-FU (10 μM)] | 0.5965 | 0.0180 | IJ |
| Oxypurinol (3.0 mM) [5-FU (10 μM)] | 0.7165 | 0.0117 | H |
| Allopurinol (0.1 mM) [5-FU (10 μM)] | 0.6303 | 0.0068 | I |
| Allopurinol (0.3 mM) [5-FU (10 μM)] | 0.5100 | 0.0102 | KLM |
| Allopurinol (1.0 mM) [5-FU (10 μM)] | 0.4370 | 0.0146 | MN |
| Allopurinol (3.0 mM) [5-FU (10 μM)] | 0.4348 | 0.0154 | MN |
| NaOH (1 mM) [5-FU (10 μM)] | 0.4880 | 0.0125 | LMN |
| NaOH (2 mM) [5-FU (10 μM)] | 0.4799 | 0.0094 | LMN |
| NaOH (3 mM) [5-FU (10 μM)] | 0.4731 | 0.0112 | LMN |
| NaOH (4 mM) [5-FU (10 μM)] | 0.5413 | 0.0182 | JKL |

¹Tukey's multi-comparison (levels not connected by same letter significantly different (p < 0.05))

The presence of allopurinol over the range of 0.1 mM to 3.0 mM in the absence of 5-FU had a small positive effect on confluent NHEK cell viability across the tested concentration range. When applied in the presence of 5-FU, allopurinol showed a protective effect for confluent NHEK cells that was generally less than oxypurinol. Cell survival decreased with increasing concentration of allopurinol over the tested concentration range.

The presence of NaOH over the range of 1 mM to 4.0 mM in the presence of 5-FU had a small positive effect on confluent NHEK cell viability across the tested concentration range, with the greatest protection being at the highest tested concentration of 3.0 mM. The protection levels, however, were significantly lower than those achieved by the oxypurinol or the allopurinol.

### Example 5

Referring to Table 5, the cell viability data for cells in the presence of 5-FU and either oxypurinol or allopurinol from Tables 2-4 was converted into a relative cell protection from 5-FU. Only data from tests done with formulations lacking adenine and uracil was included in preparing the data shown in Table 5. The relative protection was calculated as the fraction of the viability loss (assumed to be caused by 5-FU toxicity) that is prevented by the presence of the oxypurinol/allopurinol. A value of 0 would indicate the cell viability being the same as if the oxypurinol/allopurinol were not present. A value of 1 would indicate the cell viability being the same as if no 5-FU or oxypurinol/allopurinol were present. A negative relative protection would indicate a lower cell viability in the presence of 5-FU and oxypurinol/allopurinol than in the presence of 5-FU and no oxypurinol/allopurinol.

The data shows a consistent general trend that increasing the amount of oxypurinol increased the amount of protection from 5-FU toxicity, whereas increasing the amount of allopurinol either decreased or minimally changed the amount of protection from 5-FU toxicity across the range of tested concentrations. These results reflect cellular behavior in a protecting formulation free of adenine and uracil. The inconsistencies between sets of trials for HPEK/oxypurinol are believed to be related to inherent differences in cell growth and metabolism for the HPEK (proliferating versus confluent) between two different sets of trials. The highest levels of protection were provided by the oxypurinol. The highest concentration of oxypurinol tested, 3 mM, is close to the upper limit of solubility for oxypurinol in the protective formulation.

TABLE 5
Relative cell protection against 5-FU toxicity

| Cell Line, Treatment | Relative Protection |
|---|---|
| HPEK, Oxypurinol (0.03 mM), [proliferating cells] | 0.1133 |
| HPEK, Oxypurinol (0.1 mM), [proliferating cells] | 0.4163 |
| HPEK, Oxypurinol (0.3 mM), [proliferating cells] | 0.6092 |
| HPEK, Oxypurinol (0.3 mM), [confluent cells] | 0.2249 |
| HPEK, Oxypurinol (1.0 mM), [confluent cells] | 0.3039 |
| HPEK, Oxypurinol (3.0 mM), [confluent cells] | 0.4822 |
| HPEK, Allopurinol (0.3 mM) | 0.2464 |
| HPEK, Allopurinol (1.0 mM) | 0.3709 |
| HPEK, Allopurinol (3.0 mM) | 0.3092 |
| NHEK, Oxypurinol (0.1 mM) | 0.2834 |
| NHEK, Oxypurinol (0.3 mM) | 0.2941 |
| NHEK, Oxypurinol (1.0 mM) | 0.3085 |
| NHEK, Oxypurinol (3.0 mM) | 0.5142 |
| NHEK, Allopurinol (0.1 mM) | 0.3664 |
| NHEK, Allopurinol (0.3 mM) | 0.1603 |
| NHEK, Allopurinol (1.0 mM) | 0.0352 |
| NHEK, Allopurinol (3.0 mM) | 0.0314 |

The relative protection calculated in Table 5 does not take into consideration the effect of the oxypurinol or allopurinol itself on the cell viability. Referring to Table 6, the cell viability data for cells in the presence of 5-FU and either oxypurinol or allopurinol from Tables 2-4 was expressed as a ratio of cell growth in the presence of 5-FU and oxypurinol or allopurinol to cell growth in the presence of oxypurinol or allopurinol and absence of 5-FU. Only data from tests done with formulations lacking adenine and uracil was included in preparing the data shown in Table 6. The relative cell growth was calculated as the cell viability in the presence of oxypurinol/allopurinol and 5-FU divided by the cell viability in the presence of the same concentration of oxypurinol/allopurinol but in the absence of 5-FU. A value of 1 would indicate the cell viability being the same as if the 5-FU were not present (complete protection by the oxypurinol/allopurinol). A value of less than 1 would indicate the cell viability being less in the presence of 5-FU (incomplete protection by the oxypurinol/allopurinol).

The data presented in this way accounts for the observation that the presence of allopurinol and oxypurinol alone affected the cell growth and the cell viability results. Although there were some inconsistencies in the magnitude of the observed protection between the sets of trials, a consistent general trend was observed that increasing the amount of oxypurinol increased the amount of protection from 5-FU toxicity, whereas increasing the amount of allopurinol either decreased or minimally changed the amount of protection from 5-FU toxicity. These results reflect cellular behavior in a protecting formulation free of adenine and uracil. The inconsistencies between sets of trials are believed to be related to inherent differences in cell growth and metabolism between different sets of trials. The highest levels of protection were provided by the oxypurinol.

TABLE 6
Relative cell growth in the presence of 5-FU

| Cell Line, Treatment | Relative Growth |
|---|---|
| HPEK, Oxypurinol (0.03 mM), [proliferating cells] | 0.6414 |
| HPEK, Oxypurinol (0.1 mM), [proliferating cells] | 1.0000 |
| HPEK, Oxypurinol (0.3 mM), [proliferating cells] | 0.8465 |
| HPEK, Oxypurinol (0.3 mM), [confluent cells] | 0.7096 |
| HPEK, Oxypurinol (1.0 mM), [confluent cells] | 0.7013 |
| HPEK, Oxypurinol (3.0 mM), [confluent cells] | 0.9755 |
| HPEK, Allopurinol (0.3 mM) | 0.6152 |
| HPEK, Allopurinol (1.0 mM) | 0.6243 |
| HPEK, Allopurinol (3.0 mM) | 0.7231 |
| NHEK, Oxypurinol (0.1 mM) | 0.6549 |
| NHEK, Oxypurinol (0.3 mM) | 0.6492 |
| NHEK, Oxypurinol (1.0 mM) | 0.6226 |
| NHEK, Oxypurinol (3.0 mM) | 0.7323 |
| NHEK, Allopurinol (0.1 mM) | 0.6045 |
| NHEK, Allopurinol (0.3 mM) | 0.4519 |
| NHEK, Allopurinol (1.0 mM) | 0.3924 |
| NHEK, Allopurinol (3.0 mM) | 0.4086 |

FIG. 1 shows that oxypurinol works significantly better to protect keratin-forming HPEK cells from 5-FU toxicity than allopurinol across the range of tested concentrations. The HPEK cells at 3 mM of oxypurinol show unexpected preservation of growth with the addition of 10 μM of 5-FU. The relative cell viability of HPEK in the presence and absence of 5-FU is similar. The addition of 5-FU with 3 mM of oxypurinol with HPEK, however, shows increased survival compared with 1 mM of oxypurinol with 5-FU (where the control with only 1 mM of oxypurinol shows near control cell growth. If the oxypurinol were exerting a toxic effect, one might well expect decreased cell survival with both oxypurinol at 3 mM and 5-FU.

FIG. 2 shows that oxypurinol works significantly better to protect keratin-forming NHEK cells from 5-FU toxicity than allopurinol across the range of tested concentrations. Although neither oxypurinol nor allopurinol perform quite as well with NHEK cells as with HPEK cells, the difference between oxypurinol and allopurinol is even greater with NHEK cells. At 3.0 mM, the relative growth in the presence of 5-FU with oxypurinol is over 73%, whereas the relative growth in the presence of 5-FU with allopurinol is less than 41%.

Without wishing to be bound by theory, the data may suggest that the presence of 3 mM of oxypurinol in a formulation slows the growth of HPEK cells (which are primary cells), rather than killing them. There is a consistent relative reduction of cell growth of HPEK in all four wells exposed to 3 mM of oxypurinol alone in comparison to the cell replication of the immortalized cell line NHEK, which is not changed in 3 mM of oxypurinol alone.

All above-mentioned references are hereby incorporated by reference herein.

While the invention has been described with reference to one or more embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. In addition, all numerical values identified in the detailed description shall be interpreted as though the precise and approximate values are both expressly identified.

What is claimed is:

1. A protective formulation for treatment of at least one side-effect associated with administration of at least one therapeutic agent, the protective formulation comprising a therapeutic amount of oxypurinol.

2. The protective formulation of claim 1, wherein the protective formulation is free of adenine and uracil.

3. The protective formulation of claim 1, wherein the at least one therapeutic agent comprises at least one fluoropyrimidine.

4. The protective formulation of claim 3, wherein the at least one fluoropyrimidine is selected from the group consisting of capecitabine, carmofur, doxifluridine, floxuridine, 5-fluorouracil, and tegafur.

5. The protective formulation of claim 1, wherein the at least one therapeutic agent comprises at least one non-pyrimidine drug.

6. The protective formulation of claim 1, wherein the oxypurinol is present at a concentration of at least 0.01%, by weight, in the protective formulation.

7. The protective formulation of claim 1, wherein the oxypurinol is present at a concentration in the range of 1% to 50%, by weight, in the protective formulation.

8. The protective formulation of claim 1, wherein the at least one side-effect comprises hand-foot syndrome.

9. The protective formulation of claim 1, wherein the protective formulation is a topical formulation.

10. The protective formulation of claim 1, wherein oxypurinol is the only active ingredient in the protective formulation.

11. A method for treatment of at least one side-effect associated with administration of at least one therapeutic agent to a patient, the method comprising applying or locally delivering a protective formulation comprising a therapeutic amount of oxypurinol to a site of protection of the patient.

12. The method of claim 11 further comprising administering the at least one therapeutic agent to the patient.

13. The method of claim 11, wherein the at least one side-effect comprises hand-foot syndrome.

14. The method of claim 11, wherein the applying or locally delivering comprises topically administering the protective formulation and wherein the site of protection of the patient comprises palms of hands and soles of feet of the patient.

15. The method of claim 11, wherein the protective formulation is free of adenine and uracil.

16. The method of claim 11, wherein the at least one therapeutic agent comprises at least one fluoropyrimidine.

17. The method of claim 11, wherein the at least one therapeutic agent comprises at least one non-pyrimidine drug.

18. The method of claim 11, wherein the therapeutic amount of oxypurinol provides a concentration of oxypurinol at the site of protection in the range of 0.1 mM to 3 mM.

19. The method of claim 11, wherein the protective formulation is a topical formulation.

20. The method of claim 11, wherein oxypurinol is the only active ingredient in the protective formulation.
